(19) United States
(12) Patent Application Publication
(10) Pub. No.: US 2022/0387109 A1
(43) Pub. Date: Dec. 8, 2022

(54) CONNECTOME BASED NEURAL PROSTHESIS

(71) Applicant: X Development LLC, Mountain View, CA (US)

(72) Inventors: Sarah Ann Laszlo, Mountain View, CA (US); Benoit Schillings, Los Altos Hills, CA (US)

(21) Appl. No.: 17/337,020

(22) Filed: Jun. 2, 2021

(51) Int. Cl. A61B 34/10; G05B 19/4099; B33Y 50/00

(52) U.S. Cl. CPC A61B 34/10; G05B 19/4099; B33Y 50/00; G05B 2219/45172; G05B 2219/35134; G05B 2219/49023; A61B 2034/108

(57) ABSTRACT

In one aspect, there is provided a method performed by one or more data processing apparatus, the method including obtaining a baseline image of a baseline biological organism brain, obtaining a follow-up image of a target biological organism brain, wherein the follow-up image shows at least a damaged region of the target biological organism brain, processing the baseline image and the follow-up image to generate data defining a predicted anatomical microstructure of the damaged region of the target biological organism brain before the target biological organism brain was damaged, and generating a design for a neural prosthesis for replacing the damaged region of the target biological organism brain based on the predicted anatomical microstructure of the damaged region of the target biological organism brain before the target biological organism brain was damaged.

### BACKGROUND

This specification relates to designing a neural prosthesis.

Generally, a prosthesis refers to an implantable device that can at least partially restore functionality of the body that may have been lost due to injury, disease, or pathology.

### SUMMARY

This specification describes a method performed by one or more data processing apparatus for generating a design for a neural prosthesis for replacing a damaged region of the brain, e.g., that may have been damaged due to injury, disease, or pathology. The neural prosthesis can be implanted into the brain, e.g., to at least partially restore lost brain functionality. The neural prosthesis can be represented by a digital model that specifies various parameters of the prosthesis, e.g., the structure and configuration of the prosthesis.

According to a first aspect, there is provided a method including obtaining a baseline image of a baseline biological organism brain, obtaining a follow-up image of a target biological organism brain, wherein the follow-up image shows at least a damaged region of the target biological organism brain, processing the baseline image and the follow-up image to generate data defining a predicted anatomical microstructure of the damaged region of the target biological organism brain before the target biological organism brain was damaged, and generating a design for a neural prosthesis for replacing the damaged region of the target biological organism brain based on the predicted anatomical microstructure of the damaged region of the target biological organism brain before the target biological organism brain was damaged.

In some implementations, the baseline biological organism brain is of a first biological organism and the target biological organism brain is of a second biological organism.

In some implementations, the baseline biological organism brain and the target biological organism brain are of the same biological organism, and the baseline image is obtained at a baseline time point and the follow-up image is obtained at a follow-up time point later than the baseline time point and after the target biological organism brain was damaged.

In some implementations, the design for the neural prosthesis is a digital model of the neural prosthesis.

In some implementations, the baseline image and the follow-up image are diffusion tensor images, and the predicted anatomical microstructure represents connectivity between groups of neurons in the target biological organism brain.

In some implementations, the baseline image and the follow-up image are synaptic resolution images, and the predicted anatomical microstructure represents connectivity between individual neurons in the target biological organism brain.

In some implementations, the predicted anatomical microstructure is represented as a graph including multiple nodes and multiple edges. Each edge connects a pair of nodes, each node corresponds to a respective neuronal element in the target biological organism brain, and each edge connecting a pair of nodes in the graph corresponds to a connection between a pair of neuronal elements in the target biological organism brain.

In some implementations, each edge in the graph has a weight value associated with it, and the weight value is determined from the baseline and the follow-up images, and (i) based on an area of overlap between tolerance regions around each respective neuronal element of the pair of neuronal elements, or (ii) based on a strength of water diffusion in a direction along the connection between the pair of neuronal elements.

In some implementations, the neuronal element in the target biological organism brain is a neuron, and the connection between the pair of neuronal elements in the target biological organism brain is a synapse.

In some implementations, the neuronal element in the target biological organism brain is a group of neurons, and the connection between the pair of neuronal elements in the target biological organism brain is a nerve tract.

In some implementations, processing the baseline image and the follow-up image to generate data defining the predicted anatomical microstructure of the damaged region of the target biological organism brain before the target biological organism brain was damaged includes processing the baseline image to generate data defining a baseline graph, processing the follow-up image to generate data defining a follow-up graph, and applying a graph subtraction operator to the baseline graph and the follow-up graph to generate data defining the predicted anatomical microstructure of the damaged region of the target biological organism brain before the target biological organism brain was damaged.

In some implementations, applying the graph subtraction operator to the baseline graph and the follow-up graph to generate data defining the predicted anatomical microstructure of the damaged region of the target biological organism brain before the target biological organism brain was damaged includes selecting a node in the follow-up graph, determining if the same, or corresponding, node is included in the baseline graph, and based on the determination that the same, or corresponding, node is included in the baseline graph, subtracting the node from the baseline graph.

In some implementations, generating the design for the neural prosthesis for replacing the damaged region of the target biological organism brain based on the predicted anatomical microstructure of the damaged region of the target brain before the target biological organism brain was damaged includes instantiating a synthetic neuronal element in the design for the neural prosthesis for each of the of nodes, and instantiating a synthetic connection between a pair of synthetic neuronal elements in the design for the neural prosthesis for each of the edges.

In some implementations, instantiating the synthetic connection between the pair of synthetic neuronal elements in the design for the neural prosthesis for each of the edges includes instantiating a thickness of the synthetic connection in accordance with a weight value associated with each edge.

In some implementations, the method further includes providing the design for the neural prosthesis for fabrication. The neural prosthesis is fabricated based on the design.

In some implementations, the neural prosthesis is fabricated using three-dimensional printing techniques.

In some implementations, the neural prosthesis is fabricated at least partially out of carbon nanotubes.

In some implementations, after the neural prosthesis is fabricated, the neural prosthesis is implanted into the target biological organism brain.

According to a second aspect, there is provided a system including: one or more computers, and one or more storage devices communicatively coupled to the one or more computers, where the one or more storage devices store instructions that, when executed by the one or more computers, cause the one or more computers to perform the operations of the method of any preceding aspect.

According to a third aspect, there is provided one or more non-transitory computer storage media storing instructions that, when executed by one or more computers, cause the one or more computers to perform the operations of the method of any preceding aspect.

Particular embodiments of the subject matter described in this specification can be implemented so as to realize one or more of the following advantages.

The systems described in this specification can generate a neural prosthesis design that is biologically-inspired, e.g., that is based on a region of the brain that the prosthesis aims to replace. For example, if the nervous tissue in a region of the brain was damaged, the systems described in this specification can reconstruct the topological configuration of the damaged nervous tissue and generate a neural prosthesis that has the same, or corresponding, topological configuration. Accordingly, compared to a neural prosthesis with a predefined configuration, the biologically-inspired brain-specific neural prosthesis described in this specification may more effectively restore lost brain functionality when it is implanted into the brain.

The systems described in this specification can generate a connectivity graph based on nervous tissue from the nervous system of a biological organism. The connectivity graph can represent anatomical connectivity ranging from inter-neuronal connectivity (e.g., between individual neurons) to inter-regional connectivity (e.g., between groups of neurons). The design for the neural prosthesis can be specified and ultimately manufactured at the same level of resolution as the connectivity graph. When the physical neural prosthesis is implanted into the body, the points at which the neural prosthesis can be interfaced with the nervous tissue in the body can also be specified at the same level of resolution as the connectivity graph. Accordingly, the interface of the physical neural prosthesis with the biological tissue can be precisely defined, and, as a result, greatly improved when the prosthesis is implanted into the body.

Moreover, the systems described in this specification enable the design for the neural prosthesis to be tailored in line with the needs of an individual patient, and targeted in view of a particular medical condition. The prosthesis can be a priori designed for maximum benefit, reducing the need for costly, expensive, and time-consuming, development stage.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 illustrates an example of generating a neural prosthesis design for replacing a damaged region of the brain.

FIG. 2 illustrates an example physical neural prosthesis implanted into the brain.

FIG. 3 illustrates an example of applying a graph subtraction operator.

FIG. 4 is a flow diagram of an example process for generating a neural prosthesis design.

FIG. 5 is an example data flow for generating a connectivity graph based on an image of a biological brain.

FIG. 6 is a block diagram of an example computer system.

Like reference numbers and designations in the various drawings indicate like elements.

### DETAILED DESCRIPTION

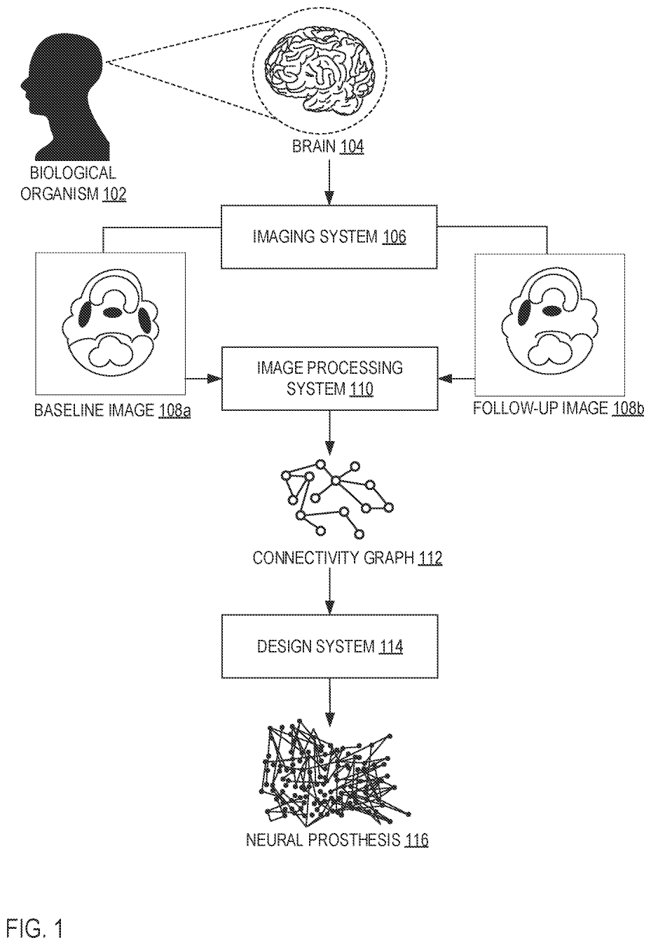

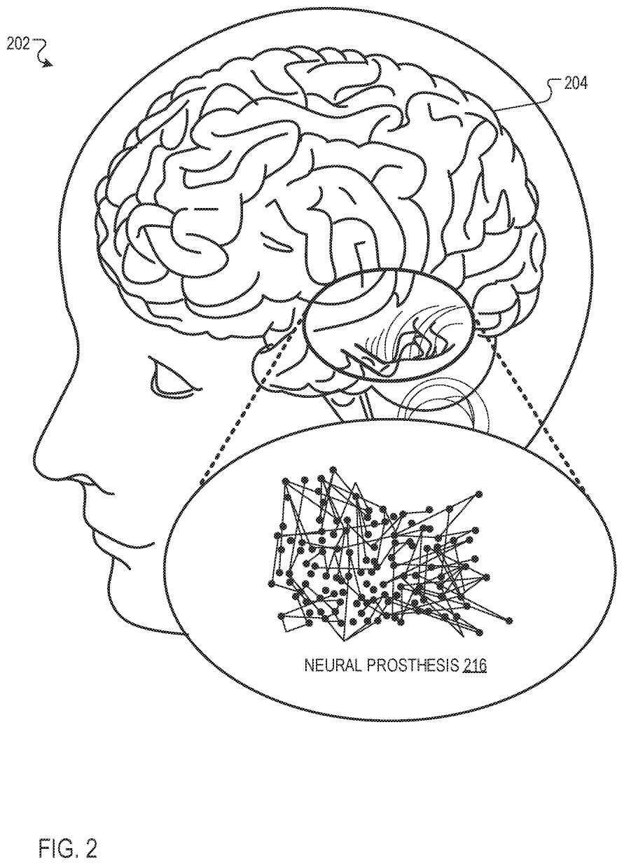

FIG. 1 illustrates an example of generating a neural prosthesis design 116 from an image of a brain 104 of a biological organism 102. Generally, as will be described below with reference to FIG. 2, a neural prosthesis 116 can be, e.g., an implantable device that can interface with a nervous tissue in the brain 104 of the biological organism 102 so as to transmit and/or modulate electrical signals. The purpose of the neural prosthesis can be, e.g., to replace damaged nervous tissue in the brain 104.

As used throughout this document, the brain 104 can refer to any amount of nervous tissue from a nervous system of the biological organism 102, and nervous tissue can refer to any tissue that includes neurons (i.e., nerve cells), synapses (i.e., connections between neurons), and/or nerve tracts (i.e., nerve fibers connecting groups of neurons). The biological organism 102 can be, e.g., a worm, a fly, a mouse, a cat, or a human. Further, as used throughout this document, a damaged brain can refer to a brain 104 that is missing any amount of nervous tissue, and/or a brain 104 that includes any amount of nervous tissue that has been damaged due to traumatic injury, non-traumatic injury, or pathology. Further, as used throughout this document, a neuronal element can refer to, e.g., a neuron, a group of neurons, or any other appropriate element, in the brain 104 of the biological organism 102.

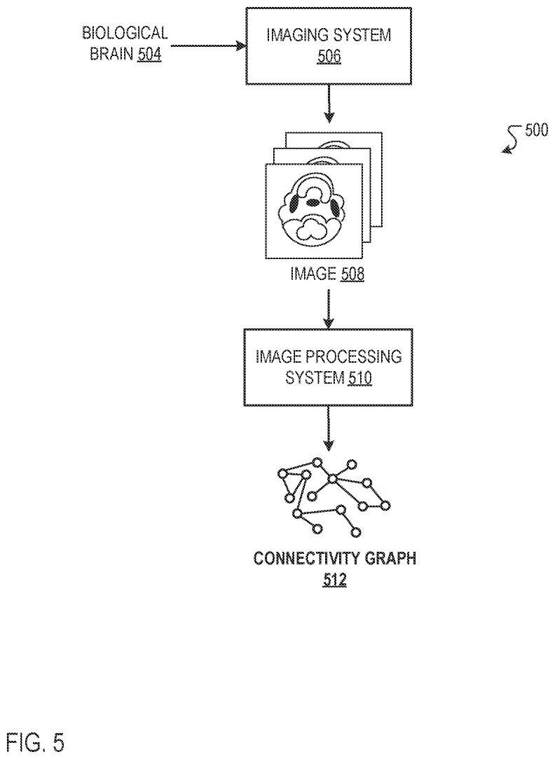

An imaging system 106 can obtain a baseline image 108a of the brain 104, and a follow-up image 108b of the brain. Example imaging systems and imaging techniques will be described in more detail below with reference to FIG. 5. The baseline image 108a can be obtained at a first point in time, and the follow-up image 108b can be obtained at, e.g., a second point in time later than the first point in time. Both images 108a, 108b can include substantially the same region (e.g., of nervous tissue) of the brain 104 of the biological organism 102.

In some implementations, the baseline image 108a is obtained from a baseline brain of a first biological organism, and the follow-up image 108b is obtained from a target brain of a second biological organism. The baseline brain can be, e.g., a brain that has not been damaged, while the target brain can be, e.g., a brain that has been damaged. For example, the first biological organism and the second biological organism can be different biological organisms, e.g., the first can be a fly, and the second can be a second, different, fly that sustained, e.g., a brain injury. In another example, the first biological organism can be a human, and the second biological organism can be a second, different, human. In yet another example, the first biological organism and the second biological organism can be the same biological organism (e.g., one and the same fly or human). In such implementations, the baseline brain and the target brain can belong to the same biological organism, e.g., can be one and the same brain, the baseline image 108a can be obtained at a baseline time point, and the follow-up image 108b can be obtained at a follow-up time point later than the baseline time point and after the biological organism sustained a brain injury.

By way of example, the biological organism 102 can be a human, the baseline image 108a can be obtained during a regular hospital check-up, and the follow-up image 108b can be obtained after the human sustained an injury (e.g., due to a stroke) that damaged at least some of the nervous tissue in the brain 104. Accordingly, the baseline image 108a can include healthy (e.g., undamaged) nervous tissue, while the follow-up image 108b can include healthy (e.g., undamaged) nervous tissue as well as damaged (or missing) nervous tissue.

An image processing system 110 can receive the baseline image 108a and the follow-up image 108b from the imaging system 106 and process the images to generate a connectivity graph 112 representing structural connectivity between neuronal elements (e.g., neurons or groups of neurons) in the nervous tissue of the brain 104, as will be described in more detail below with reference to FIG. 5. (Generally, the image processing system 110 is an example of a system which can be implemented as computer programs on one or more computers in one or more locations.) The connectivity graph 112 can represent nervous tissue in the damaged region of the brain 104 at the baseline time point.

For example, the system 110 can process the follow-up image 108b, obtained after the injury, to identify a region in the image 108b including damaged nervous tissue, and use this region as a reference to identify the same, or corresponding, region (e.g., a target region) in the baseline image 108a, obtained prior to the injury, that includes healthy (e.g., undamaged) nervous tissue. The target region of the nervous tissue in the baseline image 108a, obtained prior to the injury, can thus represent a reconstruction of the damaged nervous tissue in the follow-up image 108b obtained after the injury.

In some implementations, the system 110 can align, overlay, or otherwise compare, the baseline image 108a and the follow-up image 108b, to identify the target region of the brain in the baseline image 108a. The system 110 can process the target region of the brain 104 in the baseline image 108a to generate the connectivity graph 112 representing structural connectivity between neuronal elements (e.g., neurons, or groups of neurons) in the target region of the brain 104, e.g., in the damaged region of the brain 104 at the baseline time point. The graph 112 can be used to generate the design for the neural prosthesis 116, as will be described in more detail below with reference to FIG. 2.

Generally, the graph 112 can represent an anatomical microstructure of the nervous tissue in a region of the brain 104. An “anatomical microstructure” can refer to, e.g., the structure of synaptic connections between neurons in the brain 104, the structure of nerve tracts (e.g., nerve fibers connecting groups of neurons) in the brain 104, or the structure of any other anatomical elements discernible from the baseline image 108a and the follow-up image 108b. The graph 112 can represent anatomical connectivity ranging from inter-neuronal connectivity (e.g., between individual neurons) to inter-regional connectivity (e.g., between groups of neurons).

The graph 112 can include a set of nodes and/or a set of edges. Each node can denote a neuronal element (e.g., a neuron or a group of neurons), and each edge can denote a physical connection between two neuronal elements (e.g., a synapse or a nerve tract). Accordingly, the topological configuration of the graph 112 can define the topological connectivity pattern in the corresponding region of the brain 104.

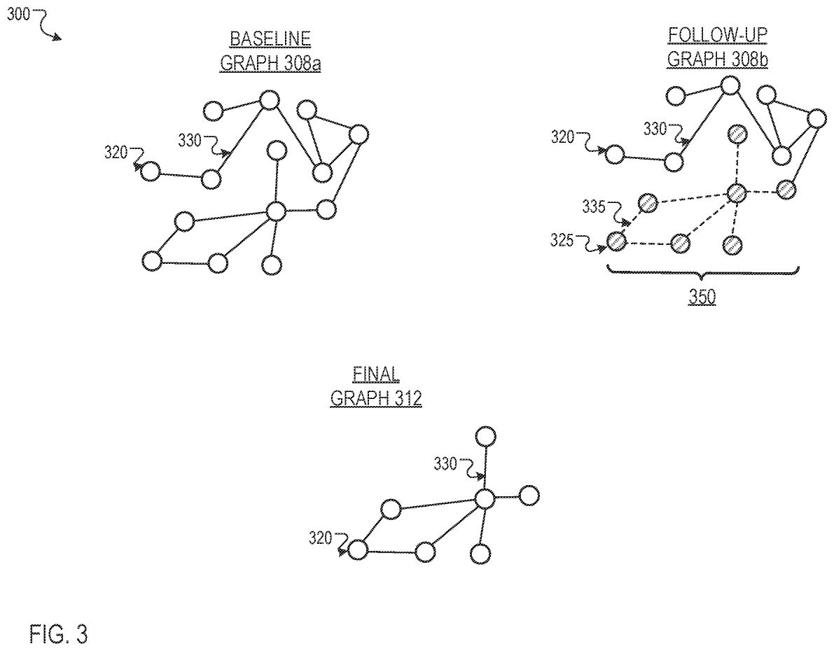

In some implementations, the image processing system 110 can generate the graph 112 without directly comparing the baseline image 108a and the follow-up image 108b, e.g., without identifying the target region in the baseline image 108a. For example, the system 110 can instead process the baseline image 108a to generate a baseline graph representing connections between neuronal elements included in the baseline image 108a, and process the follow-up image 108b to generate a follow-up graph representing connections between neuronal elements included in the follow-up image 108b. As will be described in more detail below with reference to FIG. 3, the system 110 can subtract, or otherwise compare, the baseline graph and the follow-up graph, to generate the connectivity graph 112 that represent structural connectivity between neuronal elements in the damaged region of the brain at the baseline time point.

A design system 114 can receive data defining the graph 112 and process it to generate an output. The output can be the neural prosthesis design 116, as will be described in more detail below with reference to FIG. 2. (Generally, the design system 114 is an example of a system implemented as computer programs on one or more computers in one or more locations). The neural prosthesis design 116 can be a digital model, e.g., a three-dimensional Computer-Aided Design (CAD) model.

The design 116 can be digitally represented as a synthetic structure having synthetic neuronal elements and synthetic connections between synthetic neuronal elements. Because the neural prosthesis design 116 is generated from the connectivity graph 112, it can have the same structural connectivity as the nervous tissue in the damaged region of the brain 104 at the baseline time point. The neural prosthesis design 116 can be processed in any number of ways to manufacture (e.g., fabricate) a physical neural prosthesis, which can be implanted into the brain 104 to replace damaged nervous tissue.

FIG. 2 illustrates an example physical neural prosthesis 216 implanted into the brain 204 of a human 202. The physical neural prosthesis 216 can be manufactured on the basis of a neural prosthesis design (e.g., the design 116 in FIG. 1). When implanted into the brain 204, the neural prosthesis 216 can replace nervous tissue that has been damaged due to injury or disease, and therefore at least partially restore lost brain functionality.

As described above, systems can process data, obtained from an image of the brain 204 of a biological organism 202, to generate an output that can be the neural prosthesis design (e.g., the design 116 in FIG. 1). Specifically, the image (or multiple images) can be processed to generate a connectivity graph (e.g., the connectivity graph 112 in FIG. 1) that represents the structure of connections between neuronal elements in the brain 204. The systems described in this specification can map the dataset defining the connectivity graph onto a digital representation of the neural prosthesis 216 in any variety of ways.

In some implementations, the systems can map each node in the graph, representing a biological neuronal element, onto a synthetic neuronal element. The synthetic neuronal element can be digitally represented as, e.g., a sphere. For example, as will be described in more detail below with reference to FIG. 5, each node in the graph can have a position (e.g., coordinates representing spatial location) associated with it, that corresponds to the position of the respective neuronal element in the brain 204, and the systems can instantiate the synthetic neuronal element (e.g., the sphere) at the same location as the position associated with the node.

Similarly, the systems can map each edge in the connectivity graph, representing a biological connection between two biological neuronal elements, onto a synthetic connection between two synthetic neuronal elements. The synthetic connection can be digitally represented as, e.g., a tube. For example, as will be described in more detail below with reference to FIG. 5, the dataset defining the graph can include a two-dimensional array of numerical values with a number of rows and columns equal to the number of nodes in the graph. The component of the array at position (i, j) can have value 1 if the graph includes an edge pointing from node i to node j, and value 0 otherwise. The systems can instantiate the synthetic connection (e.g., the tube) between two synthetic neuronal elements (that represent nodes i and j) when, in the dataset defining the graph, the component of the array at position (i, j) has value 1.

In some implementations, the systems can digitally represent the weight associated with each edge in the graph. The weight can be represented as, e.g., a thickness of the synthetic connection (e.g., a thickness of the tube) between two synthetic neuronal elements (e.g., between two spheres). As will be described in more detail with reference to FIG. 5, the dataset defining the graph can further include a two-dimensional array of numerical values defining the weights of each edge. More specifically, if the graph includes an edge connecting node i to node j, the component of the array at position (i, j) can have a value given by the corresponding edge weight, and otherwise the component of the array at position (i, j) can have value 0. The systems can instantiate the thickness of the tube to have a value that corresponds to e.g., the value of the edge weight of the edge that is represented by the tube. For example, a tube that represents an edge with weight value 5 can have a larger thickness than the tube that represents a second, different, edge with weight value 1.

Accordingly, the systems described in this specification can map the dataset defining the connectivity graph onto a digital representation of the neural prosthesis 216. Because the dataset is specified at the level of resolution of individual neuronal elements (e.g., neurons, or groups of neurons), the design can be digitally represented at the same level of resolution. Furthermore, when the physical neural prosthesis 216 is implanted into the brain 204, the points at which the neural prosthesis 216 can be interfaced with the nervous tissue can also be specified at the level of resolution of individual neuronal elements. In other words, an individual synthetic connection (e.g., a synthetic nerve fiber) of the neural prosthesis 216 can be interfaced with (or grafted into) an individual biological neuronal element (e.g., a group of neurons) in the brain 204.

Example techniques for interfacing synthetic neuronal elements to biological neuronal elements are described in: X. Yu et al., “Spatiotemporal characteristics of neural activity in tibial nerves with carbon nanotube yarn electrodes,” Journal of Neuroscience Methods, Volume 328, 2019, doi: 10.1016/j.jneumeth.2019.108450.

The design for the neural prosthesis can be processed to manufacture the physical neural prosthesis 216 in any variety of ways. In one example, the physical neural prosthesis 216 can be manufactured by using three-dimensional printing techniques. Synthetic neuronal elements and synthetic connections between synthetic neuronal elements can be manufactured from any appropriate materials. Example materials are described in: A. Fraczek at al., “Comparative in vivo biocompatibility study of single and multi-wall carbon nanotubes,” Acta Biomaterialia, 4, (2008), 1593-1602, doi:10.1016/j.actbio.2008.05.018; Y. Chen et al., “An active, flexible carbon nanotube microelectrode array for recording electrocorticograms,” 2011, J. Neural Eng. 8, 034001, doi:10.1088/1741-2560/8/3/034001; and R. Medupin, “Carbon Nanotube Reinforced Natural Rubber Nanocomposite for Anthropomorphic Prosthetic Foot Purpose,” Sci Rep 9, 20146 (2019). doi:10.1038/s41598-019-56778-0.

As described above, the systems can process the image (or images) of the brain to generate a connectivity graph that represents the structure of connections between neuronal elements in the brain. In some implementations, the connectivity graph can be generated by a graph subtraction operator, as will be described in more detail next with reference to FIG. 3.

FIG. 3 illustrates an example 300 of applying a graph subtraction operator to generate a connectivity graph 312. More specifically, the systems described in this specification can generate a baseline graph 308a from a baseline image of the brain obtained at a baseline time point prior to brain injury (e.g., the image 108a in FIG. 1), and generate a follow-up graph from a follow-up image of the brain (e.g., the image 108b in FIG. 1) obtained at a follow-up time point after brain injury. The graph subtraction operator can be applied to the baseline graph 308a and the follow-up graph 308b to generate the final connectivity graph 312 representing structural connectivity of nervous tissue in the damaged region of the brain at the baseline time point.

As illustrated in FIG. 3, each node in the graphs is represented by a circle (e.g., 320 and 325), and each edge in the graphs is represented by a line (e.g., 330 and 335). Generally, a node denotes a neuronal element, and an edge denotes a connection between two neuronal elements. More specifically, healthy (e.g., undamaged) neuronal elements are represented by empty circles (e.g., 320) and damaged (or missing) neuronal elements are represented by filled circles (e.g. 325). Similarly, healthy (e.g., undamaged) connections between neuronal elements are represented by solid lines (e.g., 330) and damaged (or missing) connections between neuronal elements are represented by dashed lines (e.g., 335). In this illustration, the graphs can be considered a simplified representation of connections between neuronal elements in the brain of a biological organism (actual graphs can have far more nodes and edges than depicted in FIG. 3).

The baseline graph 308a can represent the structure of connections between neuronal elements in a region of the brain that is included in the baseline image 108a. The graph 308a can include a set of nodes 320 and edges 330, representing undamaged neuronal elements and undamaged connections between them, respectively.

The follow-up graph 308b can represent the structure of connections between neuronal elements in a region of the brain that is included in the follow-up image 108b. Generally, the follow-up image 108b can include both undamaged and damaged (or missing) nervous tissue. Accordingly, a first subset of the set of nodes and edges in the follow-up graph 308b can include nodes 320 and edges 330 that represent undamaged neuronal elements and undamaged connections between them, respectively. A second subset of the set of nodes and edges in the follow-up graph 308b can include nodes 325 and edges 335 that represent damaged (or missing) neuronal elements and connections between them, respectively. For ease of reference, the subset denoting damaged nervous tissue is labeled 350 in FIG. 3.

In some implementations, the graph subtraction operator can iteratively subtract the follow-up graph 308b from the baseline graph 308a. At each iteration, the operator can randomly (or otherwise) select a node in the follow-up graph 308b (e.g., node 320) and check if the same (or corresponding) node is included in the baseline graph 308a. In one example, the same, or corresponding, node can refer to a node that has the same position (e.g., spatial location) in the dataset defining the baseline graph 308a as in the dataset defining the follow-up graph 308b.

The operator can check the spatial coordinates of the selected node from the follow-up graph 308b against the spatial coordinates of all the nodes included in the baseline graph 308a and determine if the coordinates of the selected node match any of the coordinates of the nodes included in the baseline graph 308a. If the operator determines that there is a match, then the operator determines that the selected node in the follow-up graph 308b is also included in the baseline graph 308a.

In some implementations, the graph subtraction operator can determine if the same, or corresponding, node is included in the baseline graph 308a by checking whether a difference between the spatial coordinates of the selected node from the follow-up graph 108b and the spatial coordinates of any of the nodes included in the baseline graph 108a falls within a tolerance threshold. The tolerance threshold can be, e.g., any appropriate numerical value, or a set of values. The tolerance threshold can define a limit below which a pair of nodes can be considered as “overlapping”, e.g., as having a set of spatial coordinates that are substantially similar.

The graph subtraction operator can determine the difference by, e.g., subtracting the spatial coordinates of the selected node from the spatial coordinates of each of the nodes in the baseline graph 308a. If the operator determines that the difference of the spatial coordinates any of the nodes in the baseline graph 308a and the spatial coordinates of the selected node in the follow-up graph 308b falls below the tolerance threshold, then the operator determines that the selected node in the follow-up graph 308b is also included in the baseline graph 308b.

If the same, or corresponding, node is included in the baseline graph 308a (e.g., node 320), the graph subtraction operator can subtract this node from the baseline graph 308a. In implementations where the graph subtraction operator determines whether the difference of spatial coordinates falls within the tolerance threshold, the operator can identify the node in the baseline graph 308a that has a set of spatial coordinates that falls within the threshold, with respect to the selected node in the follow-up graph 308b, and subtract this node from the baseline graph 308a. The operator can proceed by randomly selecting a new, different, node in the follow-up graph 308b.

Alternatively, if the same, or corresponding, node is not included in the baseline graph 308a, the graph subtraction operator may not delete any nodes in the baseline graph 308a, and may instead proceed by randomly selecting a new, different, node in the follow-up graph 308b. The graph subtraction operator can perform the same steps for edges in the graphs. In some implementations, at each iteration, the graph subtraction operator can randomly (or otherwise) select any number of nodes or any number of edges in the follow-up graph 308b. In some implementations, the graph subtraction operator can iteratively perform the operations until a termination criterion is satisfied. The termination criterion can be satisfied when, e.g., all nodes and all edges in the follow-up graph 308b have been selected at least once.

Instead of subtracting each individual node or edge from the baseline graph 308a, the graph subtraction operator can instead subtract a sub-graph from the baseline graph 308a. Generally, a “sub-graph” can refer to a graph specified by: (i) a proper subset of the nodes of the graph, and (ii) a proper subset of the edges of the graph. At each iteration, the operator can randomly (or otherwise) select a sub-graph (e.g., sub-graph 350) in the follow-up graph 308b and check if the same, or corresponding sub-graph is included in the baseline graph 308a. The same, or corresponding, sub-graph can refer to a sub-graph that includes the same number of nodes with the same associated positions (e.g., spatial locations), and the same number of edges with the same pattern of connectivity, in one graph as the sub-graph in a second, different graph.

As above, if the same, or corresponding, sub-graph exists in the baseline graph 308a as in the follow-up graph 308b, the graph subtraction operator can subtract the sub-graph from the baseline graph 308a. The operator can proceed by randomly selecting a new, different, sub-graph in the follow-up graph 308b. In some implementations, the graph subtraction operator can select a node, or an edge, in the follow-up graph 308b at a first iteration, and select a sub-graph in the follow-up graph 308b at a second, different, iteration.

After the termination criterion is satisfied (e.g., after all nodes and edges in the follow-up graph 308b have been selected at least once), the system 110 identifies the nodes and/or edges that have not been subtracted from the baseline graph 308a as the final graph 312. In other words, the final graph 312 includes a subset of nodes 320 and a subset of edges 330 of the baseline graph 308a. Because the final graph 312 is a sub-graph of the baseline graph 308a (which is derived from the baseline image 108a obtained at the baseline time point and before brain injury), it includes nodes 320 and edges 330 that represent undamaged neuronal elements and undamaged connections between neuronal elements (as exemplified by empty circles and solid lines in FIG. 3). In other words, the final graph 312 represents neuronal elements and connections between neuronal elements in the damaged region of the brain at the baseline time point. Accordingly, the final graph 312 represents a reconstruction of structural connectivity of nervous tissue in a region of the brain that has been damaged.

As described above with reference to FIGS. 1 and 2, a design system (e.g., the design system 114) can process data defining the final connectivity graph 312 (e.g., the graph 112) to generate an output that can be a neural prosthesis design (e.g., the design 116). The design system can map each node in the final graph 312 onto a digital representation of a synthetic neuronal element, and each edge in the final graph 312 onto a digital representation of a synthetic connection between two synthetic neuronal elements. Accordingly, the topology of the graph 312 can be mapped onto the topology of the synthetic structure representing the design for the neural prosthesis.

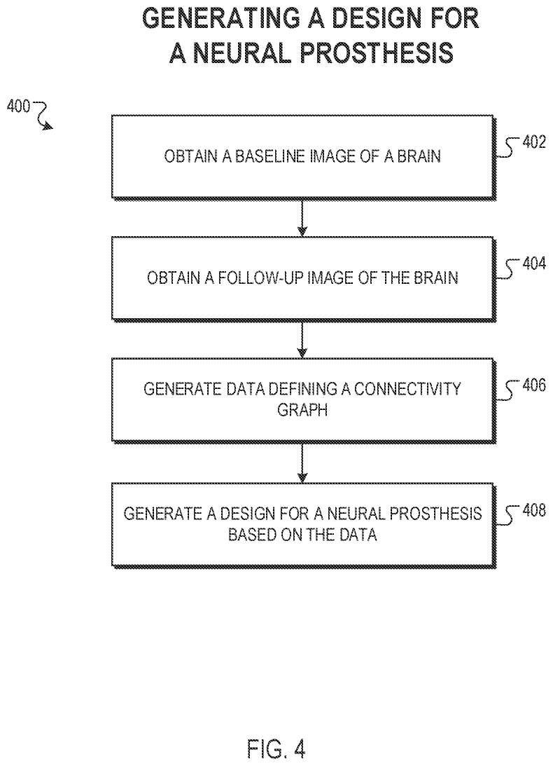

FIG. 4 is a flow diagram of an example process 400 for generating a design for a neural prosthesis. For convenience, the process 400 will be described as being performed by a system of one or more computers located in one or more locations (e.g., the imaging system 106, the image processing system 110, and/or the design system 114 shown in FIG. 1).

The system obtains a baseline image of at least a portion of a brain of a biological organism at a baseline time point (402). As described above, the baseline image of the brain can be obtained before the brain sustained an injury, e.g., at a regular hospital check-up.

The system obtains a follow-up image of at least the portion of the brain at a follow-up time point later than the baseline time point and after a region of the brain was damaged (404). Both the baseline image and the follow-up image include the region of the brain that has been damaged.

The system processes the baseline image of the brain and the follow-up image of the brain to generate data defining an anatomical microstructure of the damaged region of the brain at the baseline time point (406).

In some implementations, the system can compare the baseline image and the follow-up image. For example, the system can identify a region in the follow-up image that includes damaged nervous tissue and identify a corresponding region (e.g., target region) in the baseline image that includes undamaged nervous tissue. The system can process the target region in the baseline image to generate a connectivity graph (e.g., the anatomical microstructure) representing structural connectivity between neuronal elements in the damaged region of the brain at the baseline time point.

In some implementations, the system can generate data defining a baseline connectivity graph from the baseline image, and generate data defining a follow-up connectivity graph from the follow-up image. The system can apply a graph subtraction operator to the baseline graph and the follow-up graph to generate data defining a final connectivity graph (e.g., the anatomical microstructure) representing structural connectivity between neuronal elements in the damaged region of the brain at the baseline time point.

The system generates a design for a neural prosthesis for replacing the damaged region of the brain based on the anatomical microstructure of the damaged region of the brain at the baseline time point (408). As described above, the system can map the dataset defining the final connectivity graph (e.g., the anatomical microstructure) onto a digital representation of a synthetic structure having the same, or similar, topology as the final connectivity graph.

The system provides the neural prosthesis design for manufacturing of a physical neural prosthesis. The physical neural prosthesis can be manufactured in accordance with the design. Any variety of appropriate manufacturing techniques can be used, e.g., three-dimensional printing. After manufacturing, the physical neural prosthesis can be implanted into the brain, e.g., using any appropriate surgical techniques.

FIG. 5 illustrates an example data flow for generating a connectivity graph (e.g., representing anatomical microstructure in a brain) based on an image of the brain.

An imaging system 506 can be used to generate a high resolution image 508 of the brain 504. An image 508 of the brain 504 can be referred to as having high resolution if it has a spatial resolution that is sufficiently high to enable the identification of at least some neuronal elements (e.g., neurons, neuronal groups, nerve tracts, and/or synapses) in the brain 504. Put another way, an image 508 of the brain 504 can be referred to as having high resolution if it depicts the brain 504 at a magnification level that is sufficiently high to enable the identification of at least some neuronal elements in the brain 504. The image 508 can be a volumetric image, i.e., that characterizes a three-dimensional representation of the brain 504. The image 508 can be represented in any appropriate format, e.g., as a three-dimensional array of numerical values.

The imaging system 506 can be any appropriate system capable of generating high resolution brain images in-vivo. For example, the system 506 can be a diffusion magnetic resonance imaging (dMRI) system that measures diffusion of water molecules in the brain 504. The imaging system 506 can acquire multiple images of the brain 504, each sensitive to diffusion at a different orientation, from which a diffusion tensor for each image element (e.g., voxel) can be determined. Tractography techniques can be applied to infer the continuity of nerve fibers from voxel to voxel on the basis of tensor diffusion data. Example dMRI and tractography techniques are described with reference to: G. Gong et al., “Mapping Anatomical Connectivity Patterns of Human Cerebral Cortex Using In Vivo Diffusion Tensor Imaging Tractography,” Cerebral Cortex, Volume 19, Issue 3, March 2009, doi: 10.1093/cercor/bhn102.

In another example, the system 506 can be a diffusion spectrum MRI system that performs similar measurements as described above with reference to dMRI and further enables the reconstruction of multiple diffusion directions in each voxel. Example diffusion spectrum MM techniques are described with reference to: P. Hagmann et al., “Mapping Human Whole-Brain Structural Networks with Diffusion MM,” PLoS ONE 2(7): e597, doi: 10.1371/journal.pone.0000597.

In another example, the system 506 can be a two-photon endomicroscopy system that utilizes a miniature lens implanted into the brain to perform fluorescence imaging. This system enables in-vivo imaging of the brain at a synaptic resolution, e.g., at a resolution that enables the identification of at least some synapses and/or neurons in an image of the brain. Example techniques for generating a high resolution image of a brain using two-photon endomicroscopy are described with reference to: Z. Qin, et al., “Adaptive optics two-photon endomicroscopy enables deep-brain imaging at synaptic resolution over large volumes,” Science Advances, Vol. 6, no. 40, doi: 10.1126/sciadv.abc6521.

As described above, the image processing system 510 can process the image 508 (e.g., the baseline image 108a and the follow-up image 108b of FIG. 1) to generate a connectivity graph 512 representing structural connectivity between neuronal elements in a region of the brain 504. The graph 512 can include a set of nodes and a set of edges, such that each edge connects two nodes. To generate the graph 512, the system 510 can, e.g., identify each neuronal elements in the image 508 as a respective node in the graph, and identify each connection between a pair of neuronal elements in the image 508 as an edge between the corresponding pair of nodes in the graph.

The system 510 can identify neuronal elements (e.g., neurons, or groups of neurons) depicted in the image 508 using any of a variety of techniques. For example, the system 510 can process the image 508 to identify the positions of the neurons depicted in the image 508, and determine whether a synapse connects two neurons based on the proximity of the neurons (as will be described in more detail below).

In this example, the system 510 can process an input including: (i) the image, (ii) features derived from the image, or (iii) both, using a machine learning model that is trained using supervised learning techniques to identify neurons in images. The machine learning model can be, e.g., a convolutional neural network model or a random forest model. The output of the machine learning model can include a neuron probability map that specifies a respective probability that each voxel in the image is included in a neuron. The system 510 can identify contiguous groups of voxels in the neuron probability map as being neurons.

Optionally, prior to identifying the neurons from the neuron probability map, the system 510 can apply one or more filtering operations to the neuron probability map, e.g., with a Gaussian filtering kernel. Filtering the neuron probability map can reduce the amount of “noise” in the neuron probability map, e.g., where only a single voxel in a region is associated with a high likelihood of being a neuron.

The machine learning model used by the system 510 to generate the neuron probability map can be trained using supervised learning training techniques on a set of training data. The training data can include a set of training examples, where each training example specifies: (i) a training input that can be processed by the machine learning model, and (ii) a target output that should be generated by the machine learning model by processing the training input. For example, the training input can be a high resolution image of a brain, and the target output can be a “label map” that specifies a label for each voxel of the image indicating whether the voxel is included in a neuron. The target outputs of the training examples can be generated by manual annotation, e.g., where a person manually specifies which voxels of a training input are included in neurons.

Example techniques for identifying the positions of neurons depicted in the image 508 using neural networks (in particular, flood-filling neural networks) are described with reference to: P. H. Li et al.: “Automated Reconstruction of a Serial-Section EM Drosophila Brain with Flood-Filling Networks and Local Realignment,” bioRxiv doi:10.1101/605634 (2019).

The system 510 can identify connections between neuronal elements (e.g., synapses or nerve tracts) in the image 508 based on the proximity of the neuronal elements. For example, the system 510 can determine that a first neuron is connected by a synapse to a second neuron based on the area of overlap between: (i) a tolerance region in the image around the first neuron, and (ii) a tolerance region in the image around the second neuron. That is, the system 510 can determine whether the first neuron and the second neuron are connected based on the number of spatial locations (e.g., voxels) that are included in both: (i) the tolerance region around the first neuron, and (ii) the tolerance region around the second neuron.

For example, the system 510 can determine that two neurons are connected if the overlap between the tolerance regions around the respective neurons includes at least a predefined number of spatial locations (e.g., one spatial location). A “tolerance region” around a neuron refers to a contiguous region of the image that includes the neuron. For example, the tolerance region around a neuron can be specified as the set of spatial locations in the image that are either: (i) in the interior of the neuron, or (ii) within a predefined distance of the interior of the neuron.

In another example, the system 510 can determine whether a first group of nerves and a second group of nerves are connected by a nerve tract based on the number of spatial locations that are included in both: (i) a tolerance region around the first group of nerves, and (ii) a tolerance region around the second group of nerves. For example, the system 510 can determine that two groups of nerves are connected if the overlap between the tolerance regions around the respective groups of nerves includes at least a predefined number of spatial locations (e.g., one spatial location). A “tolerance region” around a group of nerves refers to a contiguous region of the image that includes the group of nerves. For example, the tolerance region around a group of nerves can be specified as the set of spatial locations in the image that are either: (i) in the interior of the group of nerves, or (ii) within a predefined distance of the interior of the group of nerves.

The graphing system 510 can further identify a weight value associated with each edge in the graph. For example, the system 510 can identify a weight for an edge connecting two nodes in the graph based on the area of overlap between the tolerance regions around the respective neuronal elements corresponding to the nodes in the image 508. The area of overlap can be measured, e.g., as the number of voxels in the image 508 that are included in the overlap of the respective tolerance regions around the neuronal elements.

The weight for an edge connecting two nodes in the graph can be understood as characterizing the (approximate) strength of the connection between the corresponding neuronal elements in the brain (e.g., the amount of information flow through a synapse connecting two neurons). In another example, the system 510 can identify a weight for an edge connecting two nodes based on diffusion Mill measurements. As described above, dMRI measures the strength of water diffusion in the brain and determines a diffusion tensor for each voxel in the dMRI image. The system 510 can identify the weight of a connection based on the strength of water diffusion, derived from the diffusion tensor, along the nerve tract that the connection represents.

In addition to identifying connections between neuronal elements in the image 508, the system 510 can further determine the direction of each connection using any appropriate technique. The “direction” of a connection between two neuronal elements refers to the direction of information flow between the two neuronal elements, e.g., if a first neuron uses a synapse to transmit signals to a second neuron, then the direction of the synapse would point from the first neuron to the second neuron. Example techniques for determining the directions of synapses connecting pairs of neurons are described with reference to: C. Seguin, A. Razi, and A. Zalesky: “Inferring neural signaling directionality from undirected structure connectomes,” Nature Communications 10, 4289 (2019), doi:10.1038/s41467-019-12201-w.

In implementations where the system 510 determines the directions of the connections between neuronal elements in the image 508, the system 510 can associate each edge in the graph with direction of the corresponding connection. That is, the graph can be a directed graph. In other implementations, the graph can be an undirected graph, i.e., where the edges in the graph are not associated with a direction.

The connectivity graph 512 can be represented in any of a variety of ways. For example, the graph 512 can be represented as a two-dimensional array of numerical values with a number of rows and columns equal to the number of nodes in the graph. The component of the array at position (i, j) can have value 1 if the graph includes an edge pointing from node i to node j, and value 0 otherwise. In implementations where the system 510 determines a weight value for each edge in the graph, the weight values can be similarly represented as a two-dimensional array of numerical values. More specifically, if the graph includes an edge connecting node i to node j, the component of the array at position (i, j) can have a value given by the corresponding edge weight, and otherwise the component of the array at position (i, j) can have value 0.

Further, the nodes of the connectivity graph 512 can be represented as a list, e.g., as an N×3 array specifying x, y, and z, coordinates for each of the N number of nodes in the dataset defining the connectivity graph 512. The coordinates associated with each of the nodes can define the respective spatial location of each of the neuronal elements represented by the nodes in a region of the brain. The list of nodes can be obtained, e.g., from a high resolution image of the brain by using flood-filling networks, which are described in more detail in: Peter H. Li, et al., “Automated Reconstruction of a Serial-Section EM Drosophila Brain with Flood-Filling Networks and Local Realignment,” bioRxiv doi: 10.1101/605634 (2020).

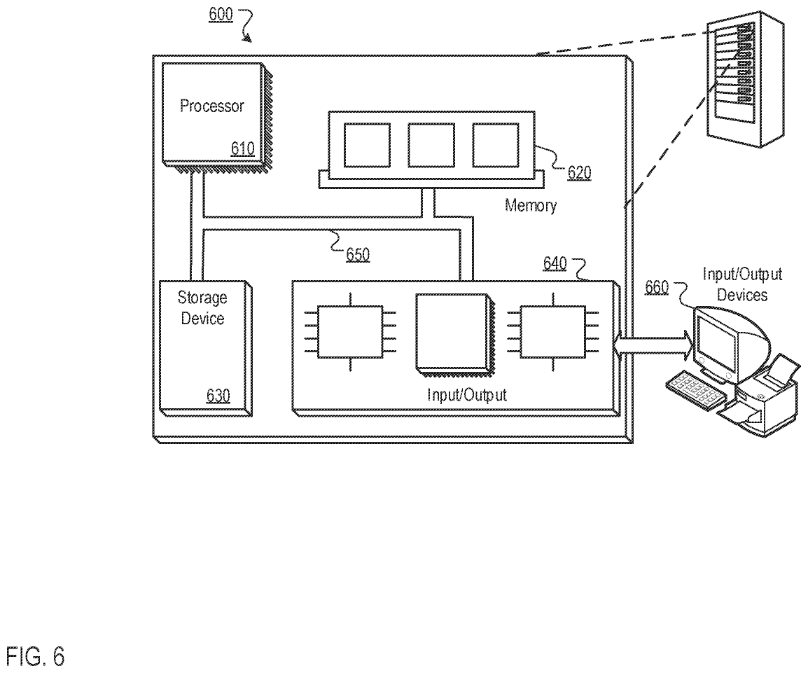

FIG. 6 is a block diagram of an example computer system 600 that can be used to perform operations described previously. The system 600 includes a processor 610, a memory 620, a storage device 630, and an input/output device 640. Each of the components 610, 620, 630, and 640 can be interconnected, for example, using a system bus 650. The processor 610 is capable of processing instructions for execution within the system 600. In one implementation, the processor 610 is a single-threaded processor. In another implementation, the processor 610 is a multi-threaded processor. The processor 610 is capable of processing instructions stored in the memory 620 or on the storage device 630.

The memory 620 stores information within the system 600. In one implementation, the memory 620 is a computer-readable medium. In one implementation, the memory 620 is a volatile memory unit. In another implementation, the memory 620 is a non-volatile memory unit.

The storage device 630 is capable of providing mass storage for the system 600. In one implementation, the storage device 630 is a computer-readable medium. In various different implementations, the storage device 630 can include, for example, a hard disk device, an optical disk device, a storage device that is shared over a network by multiple computing devices (for example, a cloud storage device), or some other large capacity storage device.

The input/output device 640 provides input/output operations for the system 600. In one implementation, the input/output device 640 can include one or more network interface devices, for example, an Ethernet card, a serial communication device, for example, and RS-232 port, and/or a wireless interface device, for example, and 802.11 card. In another implementation, the input/output device 640 can include driver devices configured to receive input data and send output data to other input/output devices, for example, keyboard, printer and display devices 660. Other implementations, however, can also be used, such as mobile computing devices, mobile communication devices, and set-top box television client devices.

Although an example processing system has been described in FIG. 6, implementations of the subject matter and the functional operations described in this specification can be implemented in other types of digital electronic circuitry, or in computer software, firmware, or hardware, including the structures disclosed in this specification and their structural equivalents, or in combinations of one or more of them.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, e.g., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which can also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program can, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, e.g., inference, workloads.

Machine learning models can be implemented and deployed using a machine learning framework, e.g., a TensorFlow framework, a Microsoft Cognitive Toolkit framework, an Apache Singa framework, or an Apache MXNet framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification includes many specific implementation details, these should not be construed as limitations on the scope of any invention or on the scope of what can be claimed, but rather as descriptions of features that can be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features can be described above as acting in certain combinations and even initially be claimed as such, one or more features from a claimed combination can in some cases be excised from the combination, and the claimed combination can be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing can be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing can be advantageous.

What is claimed is:

1. A method performed by one or more data processing apparatus, the method comprising:
obtaining a baseline image of a baseline biological organism brain;
obtaining a follow-up image of a target biological organism brain, wherein the follow-up image shows at least a damaged region of the target biological organism brain;
processing the baseline image and the follow-up image to generate data defining a predicted anatomical microstructure of the damaged region of the target biological organism brain before the target biological organism brain was damaged; and
generating a design for a neural prosthesis for replacing the damaged region of the target biological organism brain based on the predicted anatomical microstructure of the damaged region of the target biological organism brain before the target biological organism brain was damaged.

2. The method of claim 1, wherein the baseline biological organism brain is of a first biological organism and the target biological organism brain is of a second biological organism.

3. The method of claim 1, wherein the baseline biological organism brain and the target biological organism brain are of the same biological organism, and wherein the baseline image is obtained at a baseline time point and the follow-up image is obtained at a follow-up time point later than the baseline time point and after the target biological organism brain was damaged.

4. The method of claim 1, wherein the design for the neural prosthesis is a digital model of the neural prosthesis.

5. The method of claim 1, wherein the baseline image and the follow-up image are diffusion tensor images, and wherein the predicted anatomical microstructure represents connectivity between groups of neurons in the target biological organism brain.

6. The method of claim 1, wherein the baseline image and the follow-up image are synaptic resolution images, and wherein the predicted anatomical microstructure represents connectivity between individual neurons in the target biological organism brain.

7. The method of claim 1, wherein the predicted anatomical microstructure is represented as a graph comprising a plurality of nodes and a plurality of edges, wherein each edge connects a pair of nodes, each node corresponds to a respective neuronal element in the target biological organism brain, and each edge connecting a pair of nodes in the graph corresponds to a connection between a pair of neuronal elements in the target biological organism brain.

8. The method of claim 7, wherein each edge in the graph has a weight value associated with it, and wherein the weight value is determined from the baseline image and the follow-up image, and
(i) based on an area of overlap between tolerance regions around each respective neuronal element of the pair of neuronal elements, or
(ii) based on a strength of water diffusion in a direction along the connection between the pair of neuronal elements.

9. The method of claim 7, wherein the neuronal element in the target biological organism brain is a neuron, and wherein the connection between the pair of neuronal elements in the target biological organism brain is a synapse.

10. The method of claim 7, wherein the neuronal element in the target biological organism brain is a group of neurons, and wherein the connection between the pair of neuronal elements in the target biological organism brain is a nerve tract.

11. The method of claim 7, wherein processing the baseline image and the follow-up image to generate data defining the predicted anatomical microstructure of the damaged region of the target biological organism brain before the target biological organism brain was damaged comprises:
processing the baseline image to generate data defining a baseline graph;
processing the follow-up image to generate data defining a follow-up graph; and
applying a graph subtraction operator to the baseline graph and the follow-up graph to generate data defining the predicted anatomical microstructure of the damaged region of the target biological organism brain before the target biological organism brain was damaged.

12. The method of claim 11, wherein applying the graph subtraction operator to the baseline graph and the follow-up graph to generate data defining the predicted anatomical microstructure of the damaged region of the target biological organism brain before the target biological organism brain was damaged comprises:
selecting a node in the follow-up graph;
determining if the same, or corresponding, node is included in the baseline graph; and
based on the determination that the same, or corresponding, node is included in the baseline graph, subtracting the node from the baseline graph.

13. The method of claim 7, wherein generating the design for the neural prosthesis for replacing the damaged region of the target biological organism brain based on the predicted anatomical microstructure of the damaged region of the target brain before the target biological organism brain was damaged comprises:
instantiating a synthetic neuronal element in the design for the neural prosthesis for each of the plurality of nodes; and
instantiating a synthetic connection between a pair of synthetic neuronal elements in the design for the neural prosthesis for each of the plurality of edges.

14. The method of claim 13, wherein instantiating the synthetic connection between the pair of synthetic neuronal elements in the design for the neural prosthesis for each of the plurality of edges comprises:
instantiating a thickness of the synthetic connection in accordance with a weight value associated with each edge of the plurality of edges.

15. The method of claim 1, further comprising providing the design for the neural prosthesis for fabrication, wherein the neural prosthesis is fabricated based on the design.

16. The method of claim 15, wherein the neural prosthesis is fabricated using three-dimensional printing techniques.

17. The method of claim 15, wherein the neural prosthesis is fabricated at least partially out of carbon nanotubes.

18. The method of claim 15, wherein, after the neural prosthesis is fabricated, the neural prosthesis is implanted into the target biological organism brain.

19. A system comprising:
one or more computers; and
one or more storage devices communicatively coupled to the one or more computers, wherein the one or more storage devices store instructions that, when executed by the one or more computers, cause the one or more computers to perform operations including:
obtaining a baseline image of a baseline biological organism brain;
obtaining a follow-up image of a target biological organism brain, wherein the follow-up image shows at least a damaged region of the target biological organism brain;
processing the baseline image and the follow-up image to generate data defining a predicted anatomical microstructure of the damaged region of the target biological organism brain before the target biological organism brain was damaged; and
generating a design for a neural prosthesis for replacing the damaged region of the target biological organism brain based on the predicted anatomical microstructure of the damaged region of the target biological organism brain before the target biological organism brain was damaged.

20. One or more non-transitory computer storage media storing instructions that when executed by one or more computers cause the one or more computers to perform operations including:
obtaining a baseline image of a baseline biological organism brain;
obtaining a follow-up image of a target biological organism brain, wherein the follow-up image shows at least a damaged region of the target biological organism brain;
processing the baseline image and the follow-up image to generate data defining a predicted anatomical microstructure of the damaged region of the target biological organism brain before the target biological organism brain was damaged; and
generating a design for a neural prosthesis for replacing the damaged region of the target biological organism brain based on the predicted anatomical microstructure of the damaged region of the target biological organism brain before the target biological organism brain was damaged.
